(19) United States
(12) Patent Application Publication
(10) Pub. No.: US 2019/0125472 A1
(43) Pub. Date: May 2, 2019

(54) CARRYING CASE FOR VIALS

(71) Applicant: Kenneth D. Lewis, St. George, UT (US)

(72) Inventor: Kenneth D. Lewis, St. George, UT (US)

(21) Appl. No.: 16/172,556

(22) Filed: Oct. 26, 2018

Related U.S. Application Data
(60) Provisional application No. 62/577,675, filed on Oct. 26, 2017.

(51) Int. Cl. A61B 50/31; A45C 13/02; A61J 1/16

(52) U.S. Cl. CPC A61B 50/31; A61B 2050/311; A61J 1/16; A45C 13/02

(57) ABSTRACT

A carrying case for vials has a clamshell housing, at least one mounting wall comprising a plurality of shelf-retaining apertures, and a plurality of vial shelves removably attachable to the mounting wall via the shelf-retaining apertures. Each shelf has one or more vial retaining arms for securing vials of various diameters and heights thereto.

### CROSS-REFERENCE TO RELATED APPLICATIONS

This application claims the benefit of U.S. Provisional Application Ser. No. 62/577,675, filed on Oct. 26, 2017, which is incorporated herein by reference.

### TECHNICAL FIELD

The present disclosure relates to transporting and storing vials and other small bottles. More particularly, the present disclosure relates to a carrying case for transporting vials and other small bottles.

### BACKGROUND

The use of small vials and bottles has increased in recent years due, in part, to the popularity of essential oils. Medicinal vials are also used for a number of medications, such as insulin. Accordingly, it is not uncommon for the average household to have some combination of vials. However, the storage and transportation of vials is challenging, especially due to the varying sizes of bottles. In an effort to achieve a secure method of storage and transport, the prior art has used both elastic straps and polystyrene foam. Unfortunately, neither of these methods are sufficient. The elastic straps attempt to secure the vials by placing pressure on the side of the bottle, and lack a platform for the bottle to rest on. Therefore, as the elasticity wears out, the vials are no longer secured. With the polystyrene foam, receivers are shaped so as to fit a certain circumference and height of vial, which makes it impossible to fit varying sizes of circumferential vials. Likewise, because the vials are placed into polystyrene receivers, it is often difficult or impossible to see the label. Because of this problem, additional labels have placed on the lids of the vial. However, because the lids are often so small, and may be contaminated with oils, the labels to not remain properly adhered to the lid. Further, because the vials are standing in the polystyrene receivers, height is restricted to ensure that the lid can be properly secured. Therefore, if a vial is too tall, it cannot be used in the case.

Accordingly, there is a need for a carrying case for vials that can secure vials of varying circumference and height, that can display the label on the bottle, and that can be enclosed for storage or travel. Therefore, the present invention seeks to solve these and other problems.

### SUMMARY OF EXAMPLE EMBODIMENTS

In one embodiment, a carrying case for vials comprises a clamshell housing, a plurality of vial shelves removably attachable to the clamshell housing, the vial shelves comprising a platform and a plurality of vial retaining arms.

In one embodiment, the carrying case for vials comprises a closeable housing having at least one mounting wall comprising a plurality of shelf-retaining apertures, and a plurality of shelves removably attachable to the mounting wall via the shelf-retaining apertures.

In one embodiment, a carrying case for vials comprises a first wall hingedly attached to a second wall, wherein at least one wall comprises a plurality of shelf-retaining apertures, the shelf-retaining apertures configured to receive one or more locking tabs of shelves for holding vials.

In one embodiment, a carrying case for vials comprises a housing having a base with sidewalls extending upwardly therefrom, and a lid; wherein the base of the housing comprises a plurality of apertures for receiving mating protrusions from one or more shelves.

### BRIEF DESCRIPTION OF THE DRAWINGS

FIG. 1 is a perspective view of a carrying case for vials in an open position;

FIG. 2 is a rear elevation view of a carrying case for vials;

FIG. 3 is a perspective view of a shelf of a carrying case for vials;

FIG. 4 is a rear perspective view of a shelf of a carrying case for vials;

FIG. 5 is a top perspective view of a shelf of a carrying case for vials;

FIG. 6 is an exploded view of a carrying case for vials;

FIG. 7 is a side perspective view of a carrying case for vials;

FIG. 8 is a front elevation view of a carrying case for vials;

FIG. 9 is a front elevation view of a carrying case for vials;

FIG. 10 is a front elevation view of a carrying case for vials;

FIG. 11 is a rear elevation view of a mounting wall of a carrying case for vials;

FIG. 12 is a side elevation view of two mounting walls pivoted together in a closed position;

FIG. 13 is a top plan view of two mounting walls pivoted together in a closed position; and

FIG. 14 is a side perspective view of two mounting walls pivoted together in a closed position.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various different sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As will be appreciated, the carrying case for vials disclosed herein solves the need for an apparatus that can secure vials of varying circumference and height, allows the vial label to remain visible, and that can be enclosed for storage or travel.

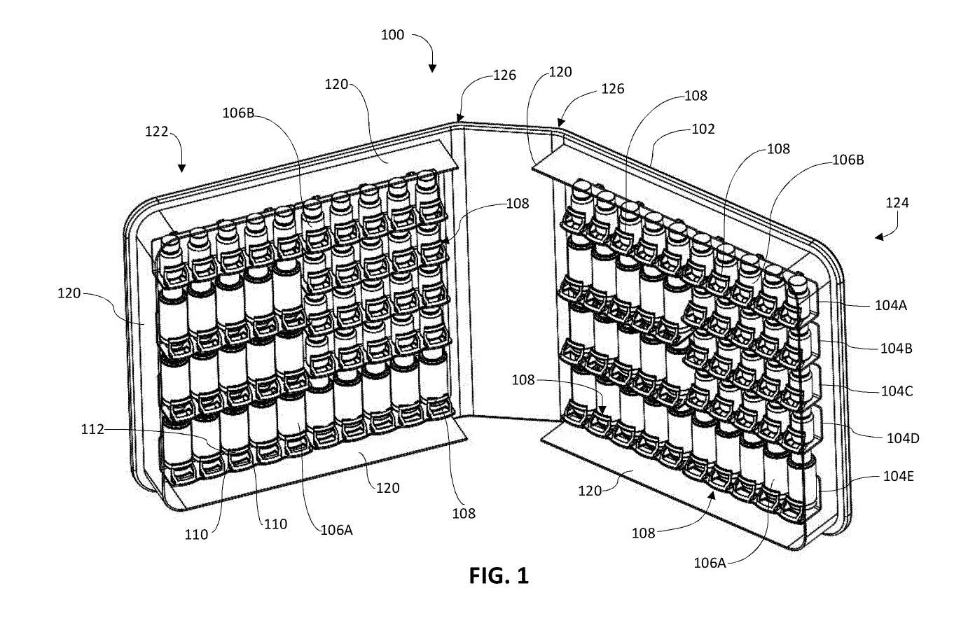

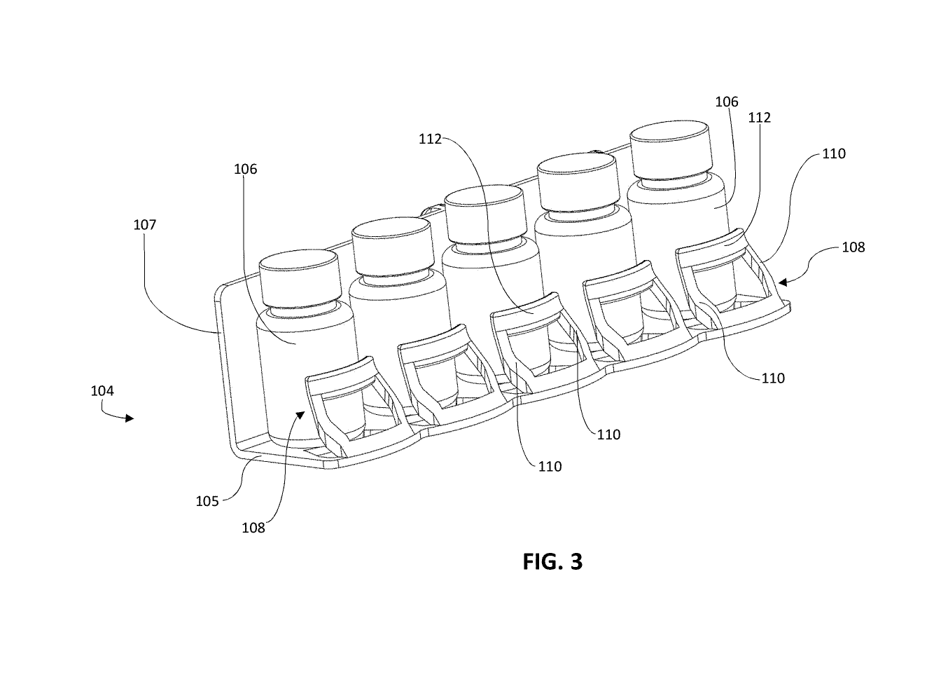

As shown in FIG. 1, in one embodiment, a carrying case for vials 100 comprises a clamshell housing 102 and a plurality of vial shelves 104A-104E. The vial shelves 104A-104E may be of varying sizes so as to accommodate different sizes of vials, such as larger vials 106A and smaller vials 106B (i.e., larger vials 106A may have a larger diameter and/or be taller than smaller vials 106B). Each vial shelf 104A-104E comprises a plurality of vial retaining arms 108. In one embodiment, as best seen in FIGS. 3-5, a vial shelf 104 comprises a platform 105, a support back 107, and vial retaining arms 108. The vial retaining arms 108 may comprise at least one vertical member 110 and a horizontal member 112. The horizontal member 112 may be curved so as to fit the outer circumference of the vial 106. While two vertical members 110 are shown on each retaining arm 108, it will be appreciated that one arm in the center could replace the two arms 110 on the sides, as well as other variations. The vial retaining arms 108 are preferably manufactured from a rigidly-flexible material allowing the vial retaining arm 108 to flex (be biased away from the support back 107) so as to allow insertion and removal of vial 106, yet restrain the vial 106 to the clamshell housing 102. Such materials may comprise plastics, rubbers, metals, or other materials. These materials may be flexible with memory properties so as to mimic a spring-biased retaining arm. In one embodiment, a spring-loaded retaining arm may be used to secure one or more vials to the shelf 104.

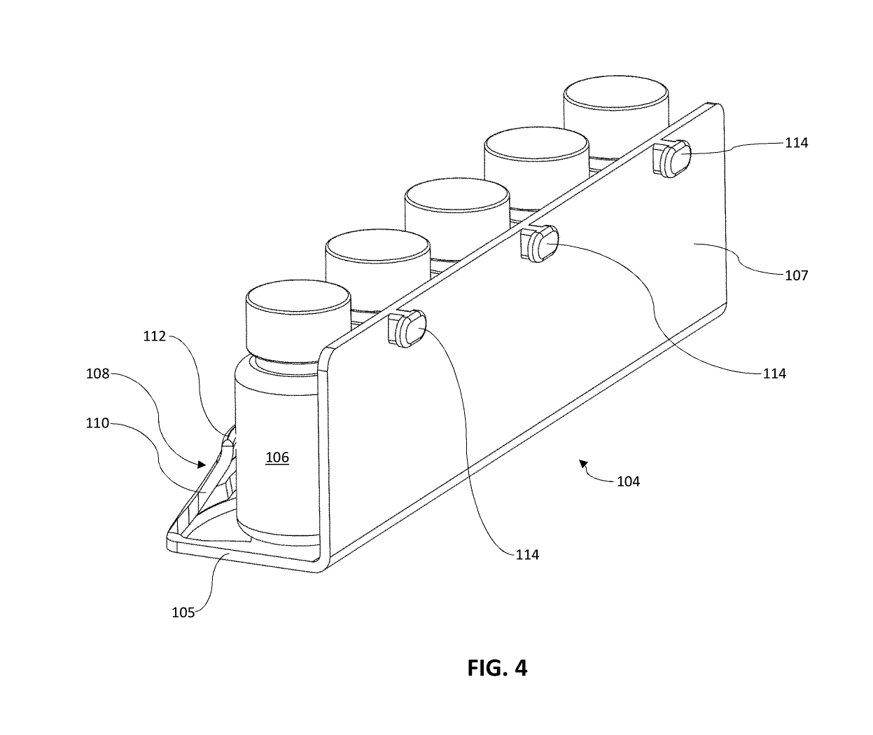

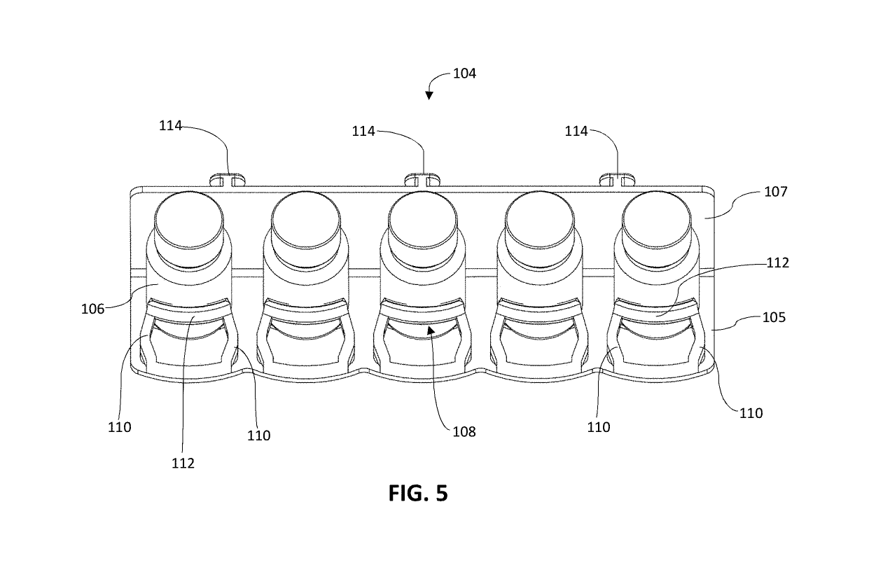

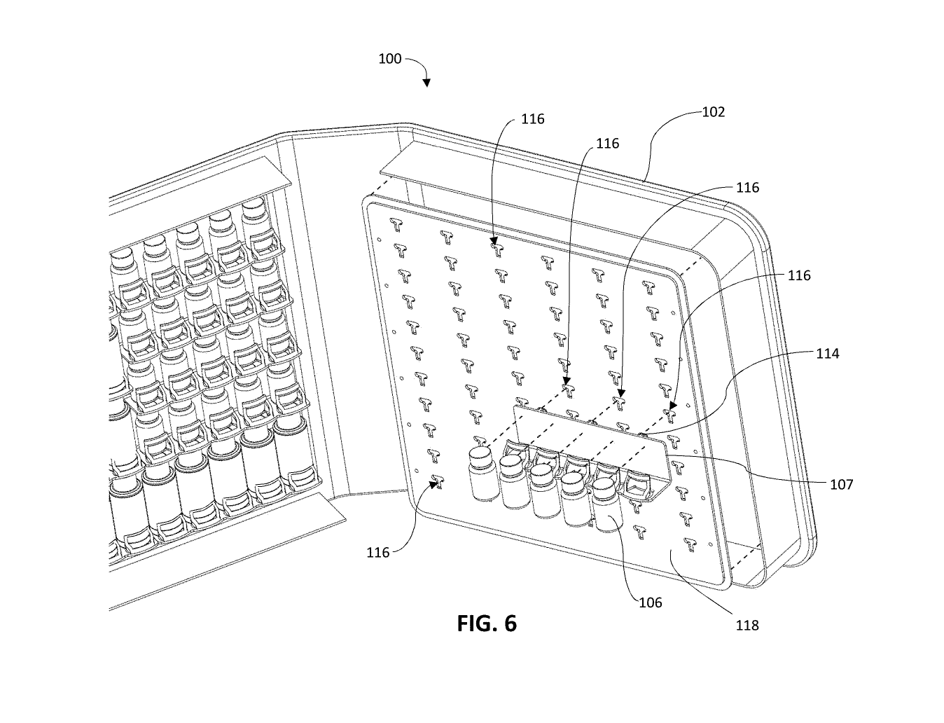

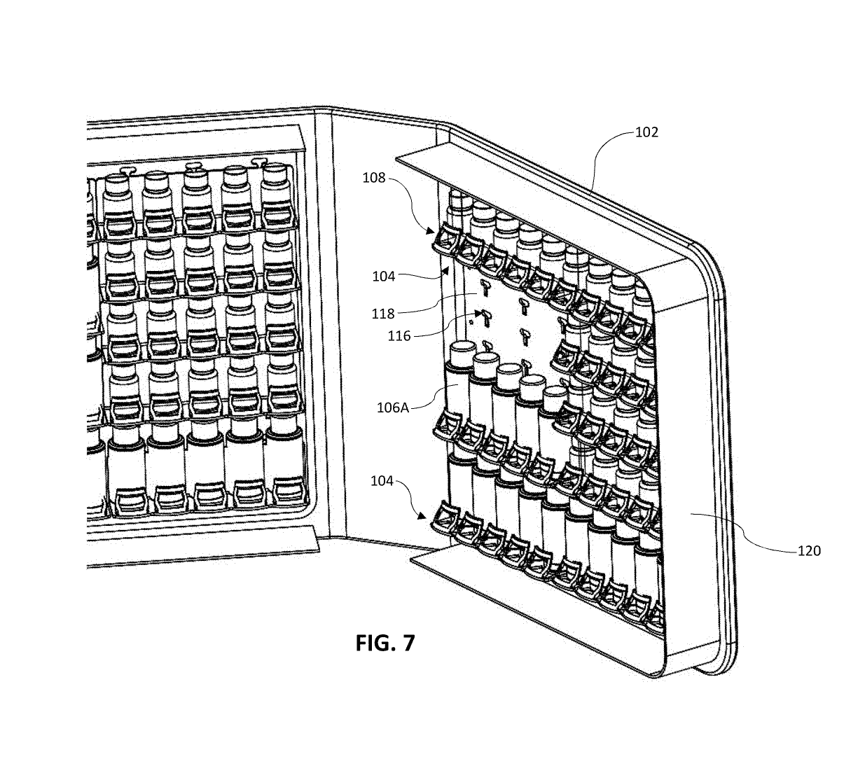

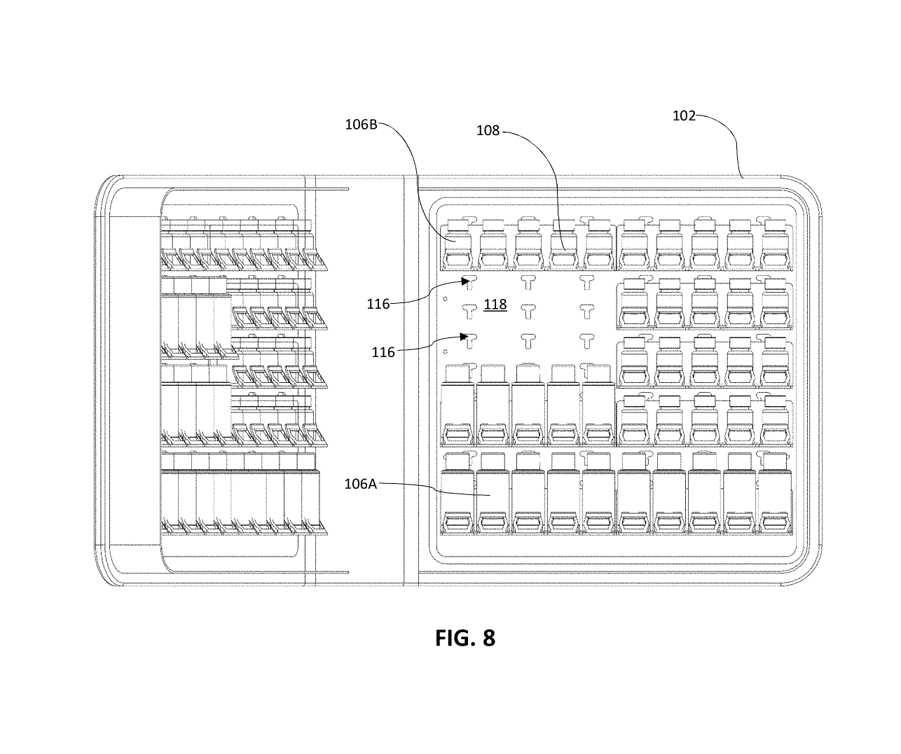

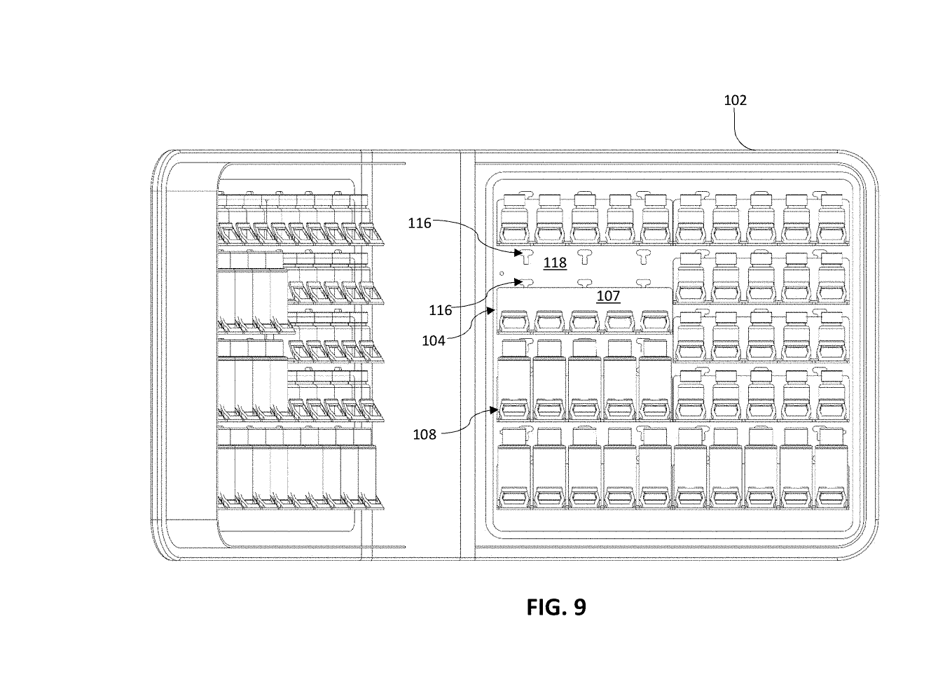

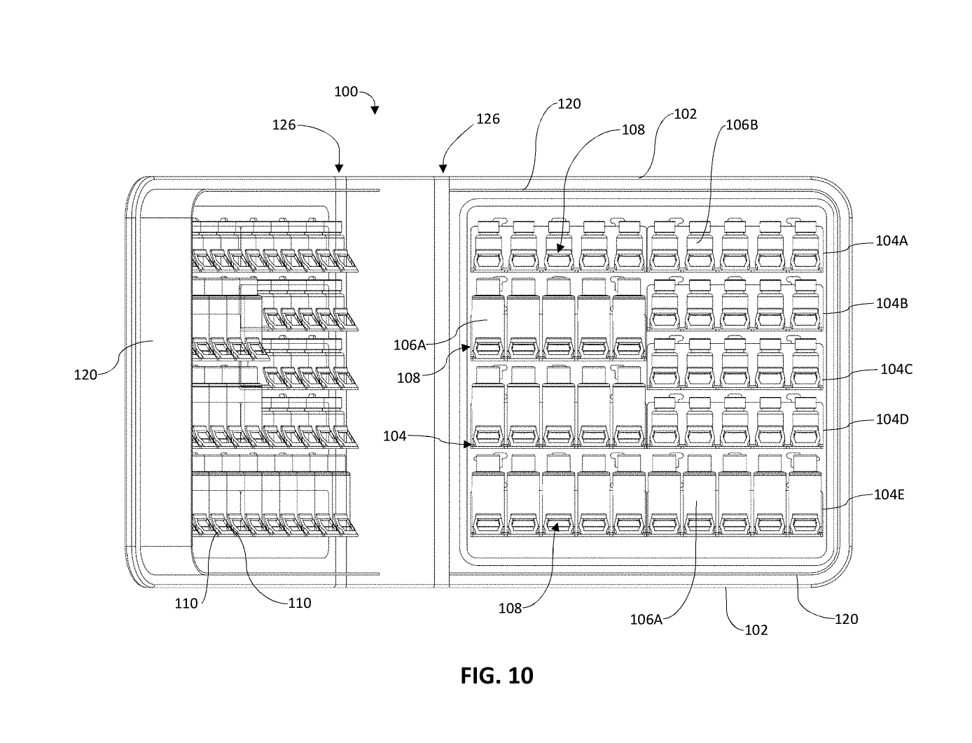

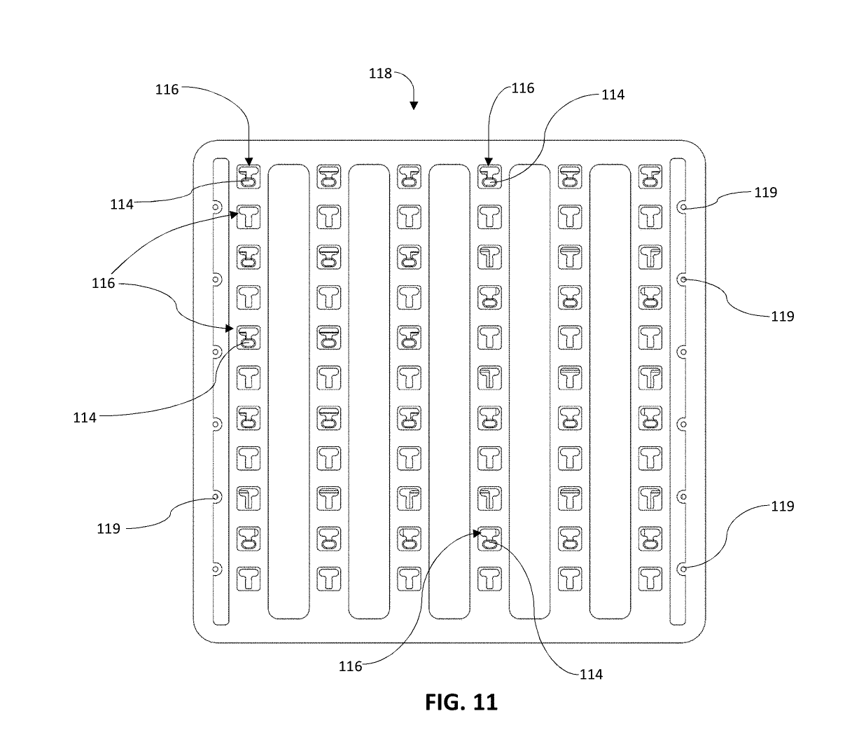

As best seen in FIGS. 4-5, each vial shelf 104 comprises locking tabs 114 on the rear side of the support back 107. The locking tabs 114 are configured to mate and interlock with the shelf-retaining apertures 116, as shown in FIGS. 6-11 of the mounting wall 118. It will be appreciated that the support back 107 is not required, and that the platform 105 may comprise the locking tabs 114 thereon, with the clamshell housing 102 functioning to support the vials 106. In other words, the vial 106 would be interposed between the vial retaining arm 108 and the clamshell housing 102. In one non-limiting example, the mounting wall 118 may have six columns and ten rows. Accordingly, a user may freely couple the vial shelves 104 in any number of positions on the mounting wall 118. FIG. 6 illustrates how the locking tabs 114 are aligned with shelf-retaining apertures 116. The ability to easily configure the vial shelves 104 allows a user to have varying heights of vials 106 stored on the vial shelves 104 without interfering with one another. The vial shelves 104 may also be of varying length, allowing a user to further customize the layout. For example, as shown in FIG. 5, the vial shelf 104 has space for five vials 106. However, in other embodiments, the vial shelf could have fewer or more (e.g., ten vial spaces to fit the example housing shown in FIG. 1). Depending upon the height of the vials 106, the number of rows will also vary. While specific examples are used above, it will be appreciated that each row may have more than five spaces or fewer than five spaces, and that the mounting wall 118 may likewise have more, or fewer, shelf-retaining apertures 116. Further, the mounting wall 118 may be a separate component from clamshell housing 102, as illustrated best in FIGS. 6 and 11. In such a scenario, the mounting wall 118 would be coupled to the clamshell housing 102 using rivets 119 (FIG. 11), adhesives, bolts, tongue and groove, or similar mechanisms. In one embodiment, the mounting wall 118 is integrated with the clamshell housing 102. In other words, the clamshell housing 102 may comprise the shelf-retaining apertures 116. In an effort to make the external side of the clamshell housing 102 more aesthetic, and to cover the shelf-retaining apertures 116, a cover may be coupled to the exterior of the clamshell housing 102.

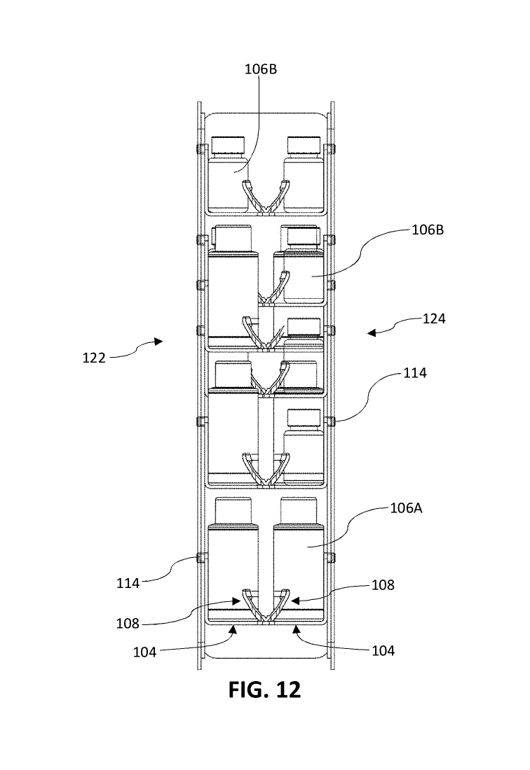

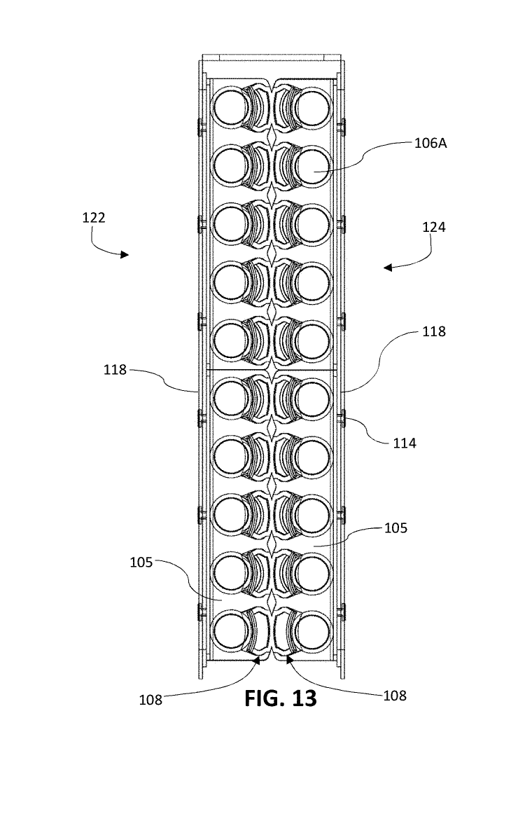

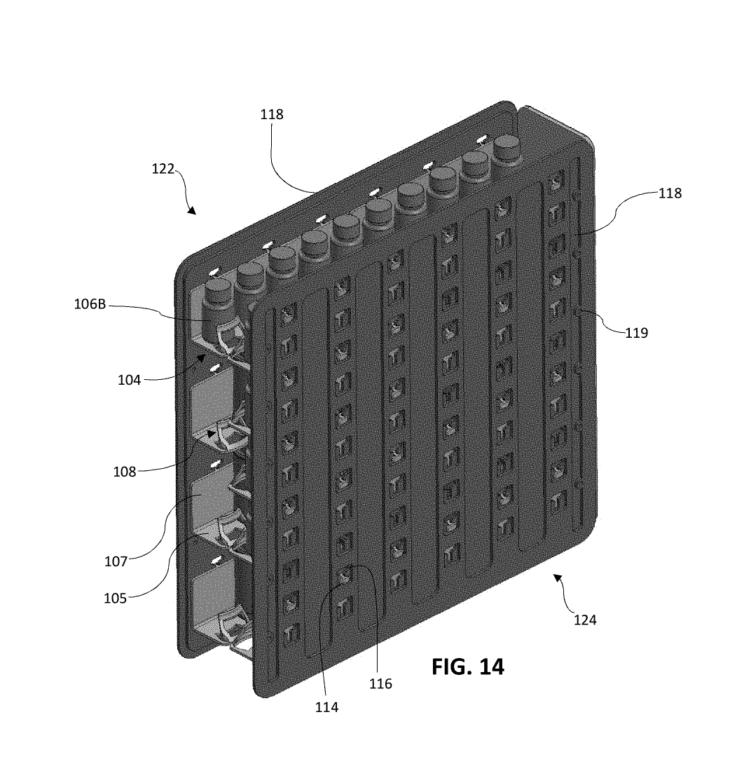

Referring back to FIG. 1, each side of the clamshell housing 102 may have a mounting wall 118 (not visible, due to the vials 106A, 106B) for holding vials 106A, 106B. An enclosing sidewall 120 may be used to prevent the vial shelves 104 and vials 106 from coming into contact with one another from the respective sides of the clamshell housing 102. The enclosing sidewall 120 may be manufactured from the same material as the clamshell housing 102 or any other, preferably rigid or semi-rigid, material, such as plastic, acrylic, metal, cardboard, carbon fiber, etc. As first clamshell end 122 pivots toward second clamshell end 124, the enclosing sidewalls 120 may overlap one another and enclose the vials 106A, 106B, which also further protects and secures them during storage and transport. FIGS. 12-14 illustrate first clamshell end 122 and second clamshell end 124 proximal to one another after pivoting, with the vials 106A, 106B interposed therebetween. The enclosing sidewalls 120 have been omitted so as to better see the interaction between the vial shelves 104 and vials 106A, 106B.

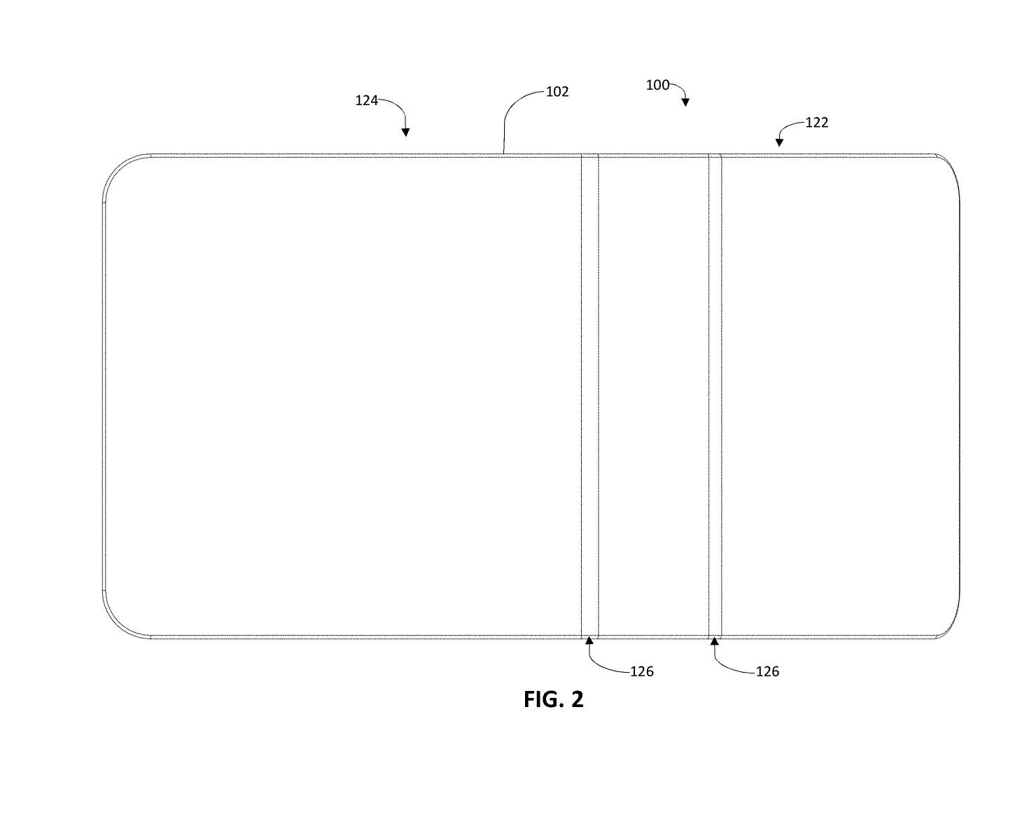

As best shown in FIGS. 1-2, the clamshell 102 may be manufactured from a single piece of material with fold points 126 facilitating the pivoting of the first clamshell end 122 and second clamshell end 124. Plastics, carboards, rubbers, and any number of materials may be used in the manufacturing so as to allow pivoting of the first and second clamshell ends 122, 124. It will be appreciated that the clamshell 102 may also have a closure securing means, such as a strap, hooks and loops, snaps, magnets, or other closure securing means.

In one method of use, a user would open the clamshell housing 102 to expose the mounting wall(s) 118. The user would then arrange the vial shelves 104 in the desired manner and secure them to the mounting wall 118 by placing the locking tabs 114 through the appropriate shelf-retaining apertures 116 and locking it in place (e.g., sliding the locking tab 114 down a neck of the shelf-retaining aperture 116). While sliding a locking tab 114 down the neck of a shelf-retaining aperture 116 is described, other locking means may also be used, such as rotating the head of the locking tab or other method. With the vial shelves 104 secured in place, a user may then place vials 106A, 106B (e.g., insulin, essential oils, etc.) on the vial shelves 104 by forcing them between the vial retaining arms 108 and the back support 107. Because the retaining arms 108 are semi-flexible, they accept the vials 106A, 106B. However, due to the memory (or spring) of the retaining arms 108, the vials 106A, 106B are securely held in place. As appreciated from the foregoing, a user may not only easily secure vials of varying sizes, but the user is also able to view the labels around the vials, which has been a considerable problem in the prior art. The user may then close the clamshell 102 and transport or store the carrying case for vials 100. While the retaining arms 108 have been described as a semi-rigid material, it may be possible, in one embodiment, to replace the retaining arms 108 with an elastic material, although this is not preferred.

While a clamshell housing 102 has been used as an example herein, it will be appreciated that the invention is not so limited. Accordingly, any type of closeable housing, whether a clamshell or a container with lid, is contemplated herein.

In one embodiment, a carrying case for vials comprises a housing having a base with sidewalls extending upwardly therefrom, and a lid for enclosing the housing. The base of the housing comprises a plurality of apertures for receiving the locking tabs 114 from one or more vial shelves 104. The base may be placed on a surface, with the lid hingedly attached or removable, with the vials secured horizontally therein. However, the base may also be placed perpendicular to the surface, such that the vials remain upright and the lid swings open horizontally.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A carrying case for vials, comprising:
a clamshell housing; and
a plurality of vial shelves removably attachable to the clamshell housing, the vial shelves comprising a platform and a plurality of vial retaining arms.

2. The carrying case for vials of claim 1, further comprising at least one mounting wall comprising a plurality of shelf-retaining apertures, the mounting wall coupleable to the clamshell housing.

3. The carrying case for vials of claim 1, wherein the clamshell housing comprises a plurality of shelf-retaining apertures and wherein the plurality of vial shelves comprise locking tabs for mating with the shelf-retaining apertures.

4. The carrying case for vials of claim 2, wherein the mounting wall comprises a plurality of shelf-retaining apertures and wherein the plurality of vial shelves comprise locking tabs for mating with the shelf-retaining apertures.

5. A carrying case for vials, comprising:
a closeable housing; and
at least one vial shelf disposed within the closeable housing, the at least one vial shelf comprising:
a platform for receiving a plurality of vials; and
a plurality of vial retaining arms, the vial retaining arms abutting the sides of the vials.

6. The carrying case for vials of claim 5, further comprising at least one mounting wall comprising a plurality of shelf-retaining apertures, the mounting wall coupleable to the closeable housing.

7. The carrying case for vials of claim 5, wherein the closeable housing comprises a plurality of shelf-retaining apertures and wherein the plurality of vial shelves comprise locking tabs for mating with the shelf-retaining apertures.

8. The carrying case for vials of claim 6, wherein the mounting wall comprises a plurality of shelf-retaining apertures and wherein the plurality of vial shelves comprise locking tabs for mating with the shelf-retaining apertures.
